SEQUENCE LISTING (2 sequences)

SEQ ID NO: 1 - DNA, 25 bp, Artificial Sequence
  synthetic oligonucleotide primer
cgrgaagctggagaaggagaagctg

SEQ ID NO: 2 - DNA, 25 bp, Artificial Sequence
  synthetic oligonucleotide primer
caagggccgcagcttacacatgttc

(19) United States
(12) Patent Application Publication
(10) Pub. No.: US 2004/0253721 A1
(43) Pub. Date: Dec. 16, 2004

(54) Methods of derivation and propagation of undifferentiated human embryonic stem (HES) cells on feeder-free matrices and human feeder layers

(76) Inventors: Ariffeen Bongso, Kent Ridge (SG); Mark Richards, Kent Ridge (SG); Fong Chui-Yee, Kent Ridge (SG); Chan Woon-Khiong, Kent Ridge (SG)

Correspondence Address: SCULLY SCOTT MURPHY & PRESSER, PC, 400 GARDEN CITY PLAZA, GARDEN CITY, NY, 11530

(21) Appl. No.: 10/811,694

(22) Filed: Mar. 29, 2004

Related U.S. Application Data
(63) Continuation of application No. PCT/AU02/01324, filed on Sep. 27, 2002.

(30) Foreign Application Priority Data
Sep. 28, 2001 (AU) PR8028
Feb. 28, 2002 (AU) PS0789
Apr. 18, 2002 (AU) PS1812
May 16, 2002 (AU) PS2364

(51) Int. Cl. C12N 5/08

(52) U.S. Cl. 435/371

(57) ABSTRACT

The present invention relates to the field of stem cell culture, in particular undifferentiated stem cell culture and to methods for derivation and propagation of such cells. More particularly, the invention relates to derivation and propagation of undifferentiated HES cells on human feeder layers and/or in the absence of a feeder layer. The human feeder layers may be selected from the group including human fetal muscle (HFM), human fetal skin (HFS), human adult fallopian tube (HAFT) fibroblasts and human adult skin cells. They may be cultured in the presence of a suitable medium selected from the group including Human Embryonic Stem Cell (HES), Knockout (KO), or Human Feeder (HF) medium supplemented with or without human serum.

The present invention relates to the field of stem cell culture, in particular undifferentiated stem cell culture and to methods for derivation and propagation of such cells. More particularly, the invention relates to derivation and propagation of undifferentiated HES cells on human feeder layers and/or in the absence of a feeder layer.

### BACKGROUND

Human pluripotent stem cells have been derived from the inner cell mass of blastocysts (embryonic stem cells) and primordial germ cells of the developing gonadal ridge (embryonic germ cells). Unlike their murine counterparts, human embryonic stem cells (HES cells) can only be maintained in culture in an undifferentiated state when grown on either γ-irradiated or mitomycin-C treated mouse embryonic fibroblast feeder cells (MEF cells).

The current absolute requirement of animal fibroblast feeders introduces significant disadvantages in the scaling up and downstream manipulation and experimentation of HES. Some of these disadvantages are (1) the potential risks of transmission of pathogens from the animal feeder cells to the human HES cells because of direct cell to cell contact and the fact that the current system of propagation (human/animal coculture) has been construed as a xenotransplant, (2) the constraints of scaling up large numbers of HES cells and their direction into specific lineages because they are mouse fibroblast dependent, (3) the labour intensiveness in having to prepare irradiated or mitomycin-C MEF each time for propagation of HES cells unlike other cells that grow on plastic, (4) the antibiotic selection of transfected HES cells would require that the fibroblast feeders also harbor similar antibiotic resistance, implying the need for creating special transgenic mouse strains with antibiotic resistance and most notably, (5) the physical proximity of MEF cells and HES cells in culture makes separation of these two cell types difficult and therefore complicates both experimental procedures and results.

The evaluation of methods for the derivation and propagation of undifferentiated HES cells on human feeder layers or in the absence of any feeder cells is therefore an urgent necessity to help overcome these disadvantages and lead onto the prospects of large/bulk scale HES cell production as well as to provide pure cultures of HES cells without the contamination of cells and proteins from other species.

Accordingly, it is an object of the present invention to overcome some of the problems of the prior art.

### SUMMARY OF THE INVENTION

In a first aspect of the present invention there is provided a method of deriving an embryonic stem (ES) cell line in a substantially undifferentiated state from an ES cell population said method comprising:

obtaining an ES cell population comprising undifferentiated ES cells;

culturing the undifferentiated ES cells on a cell support matrix in the presence of soluble factors derived from human feeder cells or equivalents thereof.

Preferably the cell line is derived from the inner cell mass of a human blastocyst.

ES cells have previously been cultured on animal/mouse fibroblast feeder layers and often the feeder layers are confined to fibroblast feeder layers. It has been found by the applicants that other human feeder layers with conventional HES medium (non-conditioned) can support prolonged HES cell growth in an undifferentiated state. Additionally, the soluble-products derived from these feeder layers (conditioned media) can also support growth of HES cells in an undifferentiated state when grown in the presence of a cell support matrix which preferably is not provided by a fibroblast feeder layer.

In yet another aspect of the present invention, there is provided a method of propagating an embryonic stem (ES) cell in a substantially undifferentiated state said method comprising:

obtaining a source of undifferentiated ES cells; and

culturing the undifferentiated ES cells on a cell support matrix in the presence of soluble factors derived from human feeder cells or equivalents thereof.

The present invention provides methods for establishing and creating new cell lines preferably derived from the inner cell masses of human blastocyst as well as providing methods which provide for continued propagation of the ensuing ES cells in an undifferentiated state. The ES cells may be obtained from an embryo, blastocyst or ICM. to establish a new cell line derived from these sources. Alternatively, the ES cells may be.obtained from an established ES cell culture and propagated under these conditions to extend the population doubling time, culturing period and passage.

The cell support matrix may be a cellular or non-cellular cell support matrix which can replace animal feeder cells. For non-cellular cell support matrices, the support may be selected from the group including, but not limited to, Collagen I, Collagen IV, human extracellular matrix or Matrigel™ or a combination thereof.

For a cellular cell support matrix, the support matrix comprises feeder cells which may be selected from the group including, but not limited to cells, human fetal muscle, human fetal skin and human adult fallopian tubal feeder fibroblast cells, human embryonic muscle, embryonic skin fibroblasts and adult skin and muscle fibroblasts.

In a preferred aspect of the present invention, there is provided a method of culturing an ES cell in a substantially undifferentiated state, said method comprising:

obtaining a source of undifferentiated ES cells;

culturing the undifferentiated cells on a non-cellular support matrix in the presence of a conditioned HES medium derived from feeder cells.

In another preferred aspect of the present invention there is provided a method. of propagating an ES cell in a substantially undifferentiated state, said method comprising:

obtaining a source of undifferentiated ES cells; and

culturing the undifferentiated cells on a non-cellular cell support matrix in the presence of a conditioned HES medium derived from human feeder cells.

Preferably, the non-cellular cell support matrix is a collagen 1 support matrix.

In another preferred aspect of the present invention there is provided a method of deriving an ES cell line in a substantially undifferentiated state from an ES cell population, said method comprising:

obtaining a source of undifferentiated ES cells; and

culturing the undifferentiated cells on a cellular cell support matrix comprising human feeder cells in the presence of a non-conditioned HES medium in the absence of LIF.

In yet another preferred aspect of the present invention, there is provided a method of propagating an ES cell in a substantially undifferentiated state, said method comprising:

obtaining a source of undifferentiated ES cells; and

culturing the undifferentiated cells on a cellular cell support matrix comprising human feeder cells in the presence of a non-conditioned HES medium in the absence of LIF.

In yet another aspect of the present invention, there is provided a cellular composition comprising proliferating undifferentiated ES cells which are free of feeder cells and wherein the cell composition is prepared by the methods described herein in the absence of feeder cells.

In yet another aspect of the present invention, there is provided a cellular composition comprising proliferating undifferentiated ES cells on a feeder cell layer wherein the cell composition is prepared by the methods described herein.

In another aspect of the present invention there is provided a human feeder cell layer which supports the derivation and culture of ES cells in a substantially undifferentiated state.

In yet another aspect of the present invention, there is provided an undifferentiated ES cell or ES cell line prepared by the methods described herein, most preferably, the ES cell or ES cell line is a mammalian ES cell Or ES cell line. More preferably it is a primate cell selected from monkey or human. Even more preferably, the ES cell or ES cell line is a human ES cell or cell line.

In another aspect of the present invention there is provided a cell culture system for deriving and culturing ES cultures in a substantially undifferentiated state, said system comprising:

a cell support matrix; and

a cell culture medium for providing soluble factors derived from human feeder cells or equivalents thereof.

In yet another preferred aspect of the present invention, there is provided a cell culture system for deriving and propagating an ES cell culture in a substantially undifferentiated state, said system comprising:

a cellular cell support matrix comprising human feeder cells; and

a non-conditioned cell culture medium absent LIF for supporting culture of the ES cells.

Thus, the present invention provides novel materials and methods for deriving and propagating ES cells in a substantially undifferentiated state. Using the methods and materials provided by the present invention, ES cells, such as those isolated from humans and monkeys, can be grown more efficiently. The ability to grow such cells without differentiation has important applications for therapeutic uses of ES cells for treating human diseases using tissue transplantation and/or gene therapy techniques.

### FIGURES

FIG. 1 shows undifferentiated HES 4 cells growing on mitomycin C treated human adult premenopausal fallopian tubal fibroblast feeder cells in the presence of HES medium. These cells are in the first passage.

FIG. 2a shows undifferentiated HES 4 cells growing on pre-coated collagen I petri dish in the presence of human adult premenopausal human fallopian tubal fibroblast conditioned HES medium. These cells are in the first passage.

FIG. 2b shows undifferentiated HES 4 cells growing on pre-coated collagen I petri dish in the presence of human adult premenopausal human fallopian tubal fibroblast conditioned HES medium. These cells are in the first passage.

FIG. 3 shows undifferentiated HES 4 cells growing on pre-coated collagen I petri dish in the presence of MEF/HES conditioned medium. These cells are in the first passage.

FIG. 4 shows undifferentiated HES 4 cells growing on pre-coated collagen I petri dish in the presence of human embryonic muscle fibroblast conditioned HES medium. These cells are in the first passage.

FIG. 5 shows undifferentiated HES 4 cells growing on pre-coated collagen I petri dish in the presence of human embryonic skin fibroblast conditioned HES medium. These cells are in the first passage.

FIG. 6 shows differentiated HES 4 cells growing on pre-coated laminin dish in the presence of MEF/HES conditioned medium. Note poor cell growth. These cells are in the first passage.

FIG. 7 shows undifferentiated HES 4 cells growing on pre-coated matrigel dish in the presence of MEF/HES conditioned medium. Note sharp colony boundary. These cells are in the first passage.

FIG. 8 shows undifferentiated HES 4 cells growing on mitomycin. C treated human fetal muscle fibroblast feeder cells in the presence of HES medium. These cells are in the first passage.

FIG. 9 shows undifferentiated HES 4 cells growing on mitomycin C treated human fetal muscle fibroblast feeder cells in the presence of HES medium. These cells are in the second passage.

FIG. 10 shows undifferentiated HES 4 cells growing on mitomycin C treated human fetal skin fibroblast feeder cells in the presence of HES medium. These cells are in the second passage.

FIG. 11 shows undifferentiated HES 3 cells growing on mitomycin C treated human adult premenopausal fallopian tubal fibroblast feeder cells in the presence of HES medium. These cells are in the second passage.

FIG. 12 shows undifferentiated HES 3 cells growing on mitomycin C treated human fetal muscle fibroblast feeder cells in the presence of HES medium. These cells are in the first passage.

FIG. 13 shows undifferentiated HES 3 cells growing on mitomycin C treated human fetal muscle fibroblast feeder cells in the presence of HES medium. These cells are in the second passage.

FIG. 14 shows undifferentiated HES 3 cells growing on mitomycin C treated human fetal skin fibroblast feeder cells in the presence of HES medium. These cells are in the second passage.

FIG. 15 shows a colony of undifferentiated HES cells (17th passage) derived and propagated from the ICM stage onwards on human fetal muscle fibroblasts.

FIG. 16 shows the edge of a colony at high magnification of undifferentiated HES cells (17^{th} passage) derived and propagated from the ICM stage onwards on human fetal muscle fibroblasts. Note that the edge of the colony has no differentiation.

FIG. 17 shows an undifferentiated human ES cell colony (5^{th} passage) propagated on HFM fibroblasts in the presence of KO-HS medium.

### DESCRIPTION OF THE INVENTION

In a first aspect of the present invention there is provided a method of deriving an embryonic stem (ES) cell line in a substantially undifferentiated state from an ES cell population said method comprising:

obtaining an ES cell population comprising undifferentiated ES cells; and

culturing the undifferentiated ES cells on a cell support matrix in the presence of soluble factors derived from human feeder cells or equivalents thereof.

The method of deriving a cell line includes creating a new cell line by a different method from a new source of ES cells preferably from the ICM stage onwards as well as being able to extend the propagation or culturing time and passage number of an already established ES cell line using this new method. When creating a new cell line from a new source of ES cells, this includes establishing a new cell line from a natural source such as, but not limited to, an embryo, a blastocyst, or inner cell mass (ICM) cells. These previously uncultured cells form the ES cell populations comprising the undifferentiated ES cells that can be further cultured to establish an ES cell line. Once established the cell line can be propagated.

In yet another aspect of the, present invention, there is provided a method of propagating an embryonic stem (ES) cell in a substantially undifferentiated state said method comprising:

obtaining an ES cell population comprising undifferentiated ES cells; and

culturing the undifferentiated ES cells on a cell support matrix in the presence of soluble factors derived from human feeder cells or equivalents thereof.

The methods of deriving and propagating may be linked by utilising the same methods which will maintain the ES cells in their undifferentiated state. Once a cell line is derived it is necessary to propagate and extend its cell culture life to enable effective scale up for large/bulk scale ES cell production.

ES cells will attach but do not grow well on tissue culture grade plastic ware and substantial differentiation often results in a short time in culture. ES cells have previously been cultured on mouse fibroblast feeder layers and often the feeder layers are confined to fibroblast feeder layers. Differentiation may occur particularly with the use of mouse fibroblast feeder layers. The mouse feeder layers often comprise heterogeneous cell populations deriving from a macerated mixture of fibroblasts from various tissues of mouse fetus. This mixture of cell products, cell types and difference of species is highly unfavourable for culturing pure ES cell populations, particularly human ES cells. It has been found by the applicants that other human feeder layers or soluble products derived from these feeder layers can support cells in an undifferentiated state when grown in the presence of a cell support matrix which preferably is not provided by a fibroblast feeder layer.

The undifferentiated embryonic stem (ES) cells may be derived from a natural source such as, but not limited to, an embryo, blastocyst; inner cell mass (ICM) or from a previous culture of ES cells which have not differentiated. Preferably, they are derived from well characterized ES cell cultures wherein the cells have been well characterized with cell markers indicative of ES cells. Suitable cell markers are provided in PCT/AU99/00990. More preferably, the ES cells are derived from cultures maintained over many passages. Hence an ES cell line from a natural source such as but not limited to, an embryo, blastocyst, or ICM will be a newly derived cell line. However, undifferentiated ES cells from already established ES cell culture may be considered to be newly derived, as used herein, when the cell line is sustained to continue propagation and increase passage numbers.

The method of propagation involves the culturing of the cell line to maintain the cell in a proliferative state which enables continued passaging.

In a preferred embodiment, the present invention provides a method of deriving an embryonic stem (ES) cell line in a substantially undifferentiated state from an ES cell population of ICM cells from a human blastocyst said method comprising:

removing inner cell mass (ICM) cells from a blastocyst wherein said ICM cells include undifferentiated ES cells; and

culturing the ICM cells including the undifferentiated ES cells on a cell support matrix in the presence of soluble factors derived from human feeder cells or equivalents thereof; and optionally

culturing to select for the undifferentiated ES cells.

ES cell lines may be derived from blastocysts resulting from a fertilized oocyte. The cells may derive from methods outlined in PCT/AU99/00990 which show cultivation of ES cells in an undifferentiated state but relies on fibroblast feeder layers. More preferably, the undifferentiated ES cell source may be obtained from a blastocyst stage of a pre-implantation stage embryo.

The blastocyst stage is obtained by fertilization of an oocyte. Preferably the embryo includes the stage after fertilisation and including up to 6 to 7 days post conception.

The embryo required in the present method may be an in vitro fertilised embryo or it may be an embryo derived by transfer of a somatic cell nucleus into an enucleated oocyte of human or non human origin which is then activated and allowed to develop to the blastocyst stage.

The embryo may be produced by fertilisation by any in vitro methods available. For instance, the embryo may be produced by fertilisation by using conventional insemination, or intracytoplasmic sperm injection. It is preferred that any embryo culture method is employed but it is most preferred that a method producing high quality (good morphological grade) blastocysts is employed. The high quality of the embryo can be assessed by morphological criteria. Most preferably the inner cell mass is well developed. These criteria can be assessed by the skilled addressee.

Following insemination, embryos may be cultured to the blastocyst stage. Embryo quality at this stage may be assessed to determine suitable embryos for deriving ICM cells. The embryos may be cultured in any medium that maintains their survival and enhances blastocyst development.

In a preferred embodiment, the blastocyst is subjected to enzymatic digestion to remove the zona pellucida or a portion thereof. Preferably the blastocyst is subjected to the digestion at an expanded blastocyst stage which may be approximately on day 6. Generally this is at approximately six days after insemination. Enzymatic digestion may be achieved using Pronase, preferably in the order of 10 IU and added to the medium in which the blastocyst is cultured.

After zona removal, and for the derivation of HES cells, the blastocyst may be washed and exposed to an anti-human whole serum which may be followed by complement treatment such as Guinea Pig complement.

Removal of the zona pellucida exposes the trophectoderm. Further removal of the trophectoderm may be utilised to expose ICM cells. The ICM cells may then be removed and cultured. Any method of removing ICM cells available to the skilled addressee may be employed as a means of retrieving undifferentiated ES cells. Preferably, the ICM may be passed through a Pasteur pipette to release and separate the ICM cells.

Optionally, the culture may comprising a heterogeneous cell population deriving from the ICM. The population of cells may contain undifferentiated ES cells. Where the ES cell source provides a heterogeneous population of cells or is an embryo, blastocyst or ICM, a further selection step may be included to either select out undifferentiated ES cells prior to culturing, or selectively culture for undifferentiated ES cells.

The ES cells may be cultured directly as part of the ICM or they may be further isolated from the ICM to obtain a substantially pure population of undifferentiated ES cells. The undifferentiated ES cells from the blastocyst may be retrieved and cultured by methods outlined in PCT/AU99/00990.

Once the ICM cells are released from the blastocyst they may be plated onto a feeder layer and subsequently subcultured when ES cell colonies become evident. This may take approximately 7 days before the first subculture.

Subculturing of the ES cells, either newly derived from a natural source or from a pre-existing ES cell culture, may be performed by cutting around the perimeter of an ES cell clump. The clump may be manually cut for propagation and preferably treated with a protease such as dispase. The ES cell clumps may then be further propagated on new feeder layers. Continued subculturing may be used in this manner to propagate the ES cells.

The newly derived ES cells are preferably mammalian ES cells, most preferably they are primate ES cells. More preferably they are human ES cells. They would be capable of maintaining an undifferentiated state when cultured under the non-differentiating conditions described herein, but have the potential to differentiate when subjected to differentiating conditions. Preferably they have the capacity to differentiate to a wide array of somatic lineages.

Instead of the source of ES cells being obtained from a natural source such as a blastocyst, the ES cells may be obtained from existing undifferentiated cultures grown on feeder cell layers. They may be removed from the feeder cells by a protease, preferably dispase or pronase and then transferred for culturing under the conditions described herein or on a cell support matrix as described herein. Culturing or propagating the ES cells in a substantially undifferentiated state is intended to provide prolonged culturing resulting in several passages of cells maintained in a substantially.undifferentiated state.

The term “substantially undifferentiated” refers to the ES cells of which at least 50% are in an undifferentiated, totipotent state. The totipotent state is a state that is capable of differentiating into any cell type including pluripotent and fully differentiated cells such as, without limitation, bone marrow stem cells, cardiac muscle cells, astrocytes or connective tissue cells.

The human fetal (HFM, HFS), adult (HAFT) feeder cells and adult skin cells are superior to the non-cellular matrices in the derivation and propagation of undifferentiated human ES cells. The ES cells may be grown on the cell support matrix in the presence of a suitable medium. A HES medium or a KO (knockout) medium may be used on feeder layers of human fetal muscle (HFM), human fetal skin (HFS), human adult fallopian tubal (HAFT) cells, adult skin cells or on a non-cellular support matrix.

HES has in its formulation 20% FCS, bovine insulin and porcine transferrin. This medium may typically contain 80% Dulbecco's Modified Eagles Medium (DMEM), 20% Hyclone defined Fetal Calf Serum (Hyclone, Logan, Utah), 1× L-Glutamine, 1× Penicillin/Streptomycin, 1× Non-essential Amino Acids (Invitrogen, Carlsbard, Calif.), 1× Insulin-Transferrin-Selenium G supplement (Invitrogen, Carlsbad, Calif.) and 1 mM β-mercaptoethanol (Sigma, St Louis, Mo.).

KO medium has in its formulation bovine insulin and porcine transferrin. This medium may typically contain 80% KNOCKOUT-DMEM (Invitrogen, Carlsbad, Calif.), 20% KNOCKOUT serum replacement (Invitrogen, Carlsbad, Calif.), 1× L-Glutamine, 1× Penicillin-Streptomycin, 1× Non-essential amino acids, 1× insulin-transferrin-selenium G supplement and 1 mM β-mercaptoethanol. 4 ng/ml rhbFGF (Sigma, St Louis, Mo.) may be added.

To reduce the possibility of the cross-transfer of animal-based pathogens from the FCS, bovine insulin and/or porcine transferrin in the HES medium or KO medium to the human fibroblast feeders, defined HES (HES-HS) or KO (KO-HS) medium may be used where the FCS is replaced with 20% human serum (HS), and the animal-based transferrin and insulin may be replaced with human insulin and human transferrin (Sigma, St Louis, Mo.). These media may be used to successfully support the undifferentiated growth of human embryonic stem cells. HES-HS and KO-HS are preferred as they have no animal-based ingredients.

The HES medium (HES-HS) supplemented with human serum (HS) may typically contain 80% DMEM, 20% pooled human serum, 1× L-Glutamine and 1× Penicillin/Streptomycin, 1× Non-essential amino acids, 1× human insulin-human transferrin-selenium G supplement and 1 mM β-mercaptoethanol.

The KO medium (KO-HS) supplemented with human serum (HS) may typically contain 80% KO DMEM, 20% pooled human serum, 1× L-Glutamine, 1× Penicillin-Streptomycin, 1× Non-essential amino acids, 1× human insulin-human transferrin-selenium G supplement and 1 mM β-mercaptoethanol. 4 ng/ml rhbFGF) may be added.

Human ICM growth for the derivation of new human embryonic stem cell lines may be supported with HFM or HFS or HAFT feeders and anyone of the following culture media (1) HES (2) KO (3) HES-HS or (4) KO-HS. HES-HS and KO-HS are preferred as they have no animal-based ingredients.

The cell support matrix may be any substance that provides substantially the same conditions for supporting cell growth as generally provided by the surfaces of feeder cells, however, the invention is not restricted only to this example. Importantly, the cell support matrix must support cell growth. The cell support matrix also supports cells in a substantially undifferentiated state.

The cell support matrix may be a cellular (feeder cells) or non-cellular cell support matrix which can replace animal feeder cells. In the latter, conditioned media may provide the necessary soluble factors for derivation and propagation of the ES cells. Conditioned media may derive from cultures of feeder cells. For non-cellular cell support matrices, the support may be selected from the group including, but not limited to, Collagen I, Collagen IV, human extracellular matrix or Matrigel™ or a combination thereof.

The various cell support matrices may be distinguished by their percentage of ES cell undifferentiation. The degree of undifferentiation may be different between the cell support matrices. Each matrix support cells which are “substantially undifferentiated” as at least 50% undifferentiation. The cell support matrix may support beyond 50% undifferentiation. Preferably, collagen 1 supports at least >90% undifferentiation. Matrigel™ may support at least >80% undifferentiation.

The thickness of the ES cell colonies may also differ between matrices. Collagen 1 colonies may be thicker than Matrigel™. Collagen 1 has been found to provide more cells and hence is superior as a support system.

Preferably the non-cellular cell support matrix is collagen I or Matrigel or a combination thereof. Most preferably, the non-cellular support matrix is collagen I or Type 1 collagen. Type I collagen is a 300 nm-long heterotrimer composed of two α₁ chains and one β₂ chain. Collagen-binding integrin receptors are α₁β₁, α₂β₁ and α₃β₁. Collagen I cellware has been used effectively for the promotion of cell attachment and spreading, cell adhesion assays and the improvement of primary cell growth in culture. Applicants have found that ES cells, particularly human ES cells attach and grow well as undifferentiated colonies on collagen I coated plasticware.

The collagen matrix may be supplied as commercially available cellware such as the BIOCOAT collagen I (5 μg/cm²) cellware petri dishes obtained from Becton Dickinson or it may be supplied in liquid form preferably at a concentration of about 5 to 10 μg/cm². The liquid form is also obtained from Becton Dickinson.

Matrigel™ comprises of laminin, collagen IV, eulactin and heparin sulfate and proteoglycan. It is obtained as precoated 35 mm dishes (cat no: 40460, Becton-Dickinson).

For a cellular cell support matrix, the cells are feeder cells and may be selected from the group including, but not limited to human adult, fetal or embryonic cells. Preferably for an adult cell, they are selected from the group including human adult fibroblast, skin, muscle or epithelial cells or any combination thereof. Preferably the adult fibroblast cell is a human adult fallopian (HAFT) fibroblast cell. Where the cell is a fetal cell, it is preferred that it is a human fetal muscle (HFM), or human fetal skin (HFS) cell; where the cell is an embryonic cell, it is preferred that it is a human embryonic muscle, or embryonic skin fibroblast cell. The adult epithelial cell is preferably an adult oviductal epithelial fibroblast.

The fetal skin and muscle fibroblasts are best obtained from the specific sites of abortuses, preferably 14 week abortuses. Adult oviductal epithelial fibroblasts may be obtained from pre-menopausal hysterectomised women. These sites are particularly useful as they provide substantially pure populations of the feeder cells. The pure populations are an advantage for derivation and propagation of undifferentiated ES cells. The feeder cells are generally either γ-irradiated or treated with mitomycin-C before use as a support matrix to the undifferentiated embryonic stem cells.

The feeder cell layers may be prepared by any method available to the skilled addressee. Human adult fallopian tubal feeder cells may be prepared by the method outlined in Bongso et al (1994, January) The growth of inner cell mass cells from human blastocysts, Theriogenology (USA), 41: 167 (Abstract); Bongso et al (1994 October) Isolation and culture of inner cell mass cells from human blastocysts, Hum Reprod (UK), 9: 2110-2117 and Bongso et al (1989) Establishment of human ampullary cell cultures, Hum Reprod, 4: 486-494.

When human adult fallopian tubal cells are first grown from donated fallopian. tubes the inner epithelial lining cells are used. The first growth of cell layers (primary culture) are epithelial (non-fibroblastic) in morphology and not stromal (fibroblastic) cells. In subsequent subcultures the epithelial cells transform into fibroblastic cells. This is quite unlike the murine embryonic fibroblasts (MEF) conventionally used where from the beginning the primary cultures are fibroblasts. Also, the adult human tubal feeder cells release tubal-specific glycoproteins possibly not released by MEF cells.

As regards to the human embryonic muscle and skin these may be collected specifically from these areas from 14 week old normal non-pathological human abortuses and grown as culture. In primary culture the embryonic muscle starts off as fibroblasts and the embryonic skin as epithelial cells. With subsequent subculturing the embryonic skin cells transform to fibroblasts. In contrast the MEF cells are fibroblasts grown from macerated murine embryos which are very small and hence a mixture of many cell types, not specifically skin and muscle.

Human adult skin feeder cells may be derived from the epidermis layer of abdominal skin via biopsies. However, the skin may be obtained from other sites or may be obtained from commercial sources which are freely available.

Feeders layers used in the present invention preferably do not require the presence of LIF. More importantly, as described below, non-conditioned culture media required to derive and propagate the ES cells in an undifferentiated state does not utilise LIF in the culture media. An advantage of the present invention is that the ES cells, particularly human ES cells are grown in the presence of human feeder cells or in the absence of feeder cell layers and hence are not xenotransplants.

The type of medium which is suitable to support a feeder layer will ensure attachment to the support matrix such as a plastic tissue culture dish. Human feeder (HF) culture medium may facilitate HFM, HFS and HAFT fibroblast feeder attachment to tissue culture plates or plastic. Generally HF medium comprises 90% Dulbecco's Modified Eagles Medium (DMEM) (Invitrogen, Carlsbard, Calif.), 10% Fetal Bovine Serum (Invitrogen, Carlsbard, Calif.), 1× L-Glutamine (Invitrogen, Carlsbard, Calif.) and 1× Penicillin/Streptomycin (Invitrogen, Carlsbard, Calif.).

To reduce the possibility of the cross-transfer of animal-based pathogens from the FBS in the HF medium to the human fibroblast feeders, FBS may be replaced with 10% pooled Human Serum (HS) and be used to successfully allow the attachment of human feeder fibroblasts to plastic. This human feeder support medium (HF-HS medium) may comprise 90% Dulbecco's Modified Eagles Medium (DMEM), 10% pooled human serum, 1× L-Glutamine and 1× Penicillin/Streptomycin. Human serum was obtained by centrifuging blood samples from patients at 300 g for 15 min. The supernatant was removed from each centrifuged blood sample and pooled. The human serum can be used fresh or after storage at 4° C. Human feeder fibroblasts can be made to attach to plastic and their growth supported with either HF or HF-HS medium. HF-HS medium is preferred, as it has no animal-based ingredients.

A further alternative medium that may be useful for the establishment of primary cultures of human fetal muscle, human fetal skin and human adult fallopian tubal cells is a human feeder establishment (HFE) medium. This medium may typically comprise 50% DMEM (Invitrogen), 50% human serum (preferably screened for HIV 1 & 2, hepatitis B)(preferably in house or commercial from Irvine SC, Calif.), 1× L-glutamine, 1× penicillin -streptomycin, 1× non-essential amino acids (Invitrogen), 1 mM mercaptoethanol and human insulin-transferrin-selenium supplement (Sigma, Mo.).

To further maintain the cells for serial passaging, a human maintenance (HM) medium may be used. This media may typically contain 80% DMEM, 20% human serum, 1× L-glutamine and 1× penicillin-streptomycin. To passage the feeder layer, trypsin is generally used to detatch the cells prior to passage. However, trypsin is generally obtained from non-human sources. Therefore it is preferred to adopt other methods to detatch the cells. It is preferred to treat the cells with EDTA with mechanical agitation or use a rubber policeman to dislodge the cells. The cells then may be maintained in the HM media.

Where the cell support matrix is a feeder layer as described above, the feeder layer may be plated directly on plain plastic or gelatin coated plastic.

Without being limited by theory, where the support matrix is a feeder cell layer, it is postulated that these feeder cells will produce their own soluble factors to support the undifferentiated growth of the ES cells. Preferably in this situation, the cells are grown in a non-conditioned medium. Preferably, the non-conditioned medium is HF, HF-HS or HM medium.

The term “soluble factors” as used herein is meant to include all factors produced or expressed by the feeder cells and which include factors that can induce cell to cell interactions and may or may not be internalized or taken up by the cell membrane but are still capable of transmitting signals through the cell membrane. Generally, the soluble factors that are produced by the cell can be internalized or solubilized in the medium and cell membranes which cause the cell to respond.

The invention also includes within its scope, the use of a non-cellular matrix, as described above, along with the feeder cells which may be plated onto the non-cellular matrix. The feeder cells provide an in situ production of soluble factors for sustaining proliferation and cultivation of the undifferentiated cells.

Most preferably the feeder cells are selected from the group including, but not limited to, human adult, fetal or embryonic cells or a combination thereof.

Preferably, for a human adult cell, the cell is selected from the group including human fibroblast, skin, muscle cells or epithelial cells. Most preferably, the human fibroblast cell is a human adult fallopian tubal (HAFT) fibroblast cell.

Most preferably the human epithelial cell is a human oviductal epithelial fibroblast cell.

Preferably for a human fetal cell, the cell is selected from the group including HFM and HFS. For a human embryonic cell, the cell is preferably a human embryonic muscle (HEM) or human embryonic skin cell.

In a further preferred embodiment the human fetal skin feeder cells are the normal human fetal skin fibroblast cell line, Detroit 551 (ATCC catalog number CCL-110).

Feeder cells may also be selected from the group including the normal fetal lung tissue cell line MRC-5 having accession number ATCC No X-55 or ATCC CCL-171 or the embryonic lung tissue cell line WI-38 having accession number ATCC-CCL-75 or ATCC-CCL-75.1.

In a further preferred aspect of the present invention, there is provided a method of deriving an ES cell line in a substantially undifferentiated state from an ES cell population, said method comprising:

obtaining an ES cell population comprising undifferentiated ES cells; and

culturing the undifferentiated cells on a non-cellular cell support matrix in the presence of a medium supplemented with conditioned media derived from human feeder cells.

In another preferred aspect of the present invention there is provided a method of propagating an ES cell in a substantially undifferentiated state from an ES cell population, said method comprising:

obtaining an ES cell population comprising undifferentiated ES cells; and

culturing the undifferentiated cells on a non-cellular cell support matrix in the presence of a medium supplemented with conditioned media derived from human feeder cells.

Preferably, the non-cellular cell support matrix is a collagen 1 support matrix.

The soluble factors or equivalents thereof are derived from human feeder cells. They are generally derived from medium in which feeder cells are grown, otherwise known as conditioned medium. This may be obtained by culturing any of the feeder cell types described above in normal medium appropriate for the cell type for a period which provides a substantially confluent monolayer and removing the cells for treatment with γ-irradiation or mitomycin-C. The inactivated cells may then be replaced and cultured in the presence of a medium which supports ES cell growth. The medium may be selected from the group including HES, KO, HES-HS or KO-HS. After a period, the culture medium may be collected as conditioned medium. Preferably, the medium is collected after a period of approximately 10 to 20 hours after plating, more preferably, the medium is collected approximately 16 hours after plating. The medium may be processed to maintain sterility. Processing methods are those known to the skilled addressee. Filtration is preferably used. The medium may be used directly. Conditioned medium could also be prepared in a similar way with confluent human monolayers that are not inactivated with mitomycin C or irradiation and such conditioned media also support HES cell growth in an undifferentiated state when grown on collagen 1 or matrigel matrices.

The term “equivalent thereof” as applied to the soluble factor means any synthetic combination of factors equating to the soluble factors found in media derived from feeder cells.

Conditioned medium may also be derived from cultured fibroblasts having been cultured in HF, HF-HS or HFE media. Supernatant media obtained from these cultures and processed under sterile conditions and described above may also be used. However, these media are best used to culture feeder layers which may then be cultured in the presence of media best suited to support ES cell growth such as, but not limited to, HES, KO, HES-HS and KO-HS. Hence, “medium supplemented with conditioned media derived from human feeder cells” may be ES cell growth medium including HES, KO, HES-HS or KO-HS media in which feeder cells have grown or it may be ES cell growth medium supplemented with HF or HF-HS in which feeder cells have grown.

Not wishing to be bound to any theory, it is believed that the use of such feeder cells, or conditioned media derived from such feeder cells, provides one or more substances necessary to promote the growth of the ES cells and/or prevent or decrease the rate of differentiation of such cells. Such substances are believed to include membrane-bound and/or soluble cell products that are secreted into the surrounding medium by the cells. In addition, those of skill will also recognize that one or more substances produced by the feeder cells, or contained in the conditioned media, can be identified and added to the cell culture media of the invention to obviate the need for such feeder cells and/or such conditioned media.

Unlike mouse feeder layers, human feeder layers, particularly pure feeder layers, do not require the presence of LIF supplemented into the medium. Without being limited by theory, the simplicity of the culture medium without LIF and the use of the human fetal and adult feeders is important for the derivation and continued propagation of the ES cells, in particular HES cells in their undifferentiated state.

Feeder cells used to produce the conditioned medium may be selected from the group including Detroit 551, MRC-5 or WI-38.

Throughout the description and claims of the specification the word “comprise” and variations of the word, such as “comprising” and “comprises”, is not intended to exclude other additives, components, integers or steps.

Culturing conditions are normal culturing conditions familiar to the skilled addressee. Temperatures of 37° C. and 5% CO₂ in air are generally adopted. However, deviations may be made from these conditions to suit the specific cell growth.

The ES cells may be propagated indefinitely. However, colony formation begins to appear after approximately 5 to 7 days. Preferably, colony formation starts at 5 days.

In another preferred aspect of the present invention there is provided a method of deriving an ES cell line in a substantially undifferentiated state from an ES cell population, said method comprising:

obtaining an ES cell population comprising undifferentiated ES cells; and

culturing the undifferentiated cells on a cellular cell support matrix comprising human feeder cells in the presence of a non-conditioned medium which supports ES cell growth in the absence of LIF.

In yet another preferred aspect of the present invention, there is provided a method of propagating an ES cell in a substantially undifferentiated state from an ES cell population, said method comprising:

obtaining an ES cell population comprising undifferentiated ES cells; and

culturing the undifferentiated cells on a cellular cell support matrix comprising human feeder cells in the presence of a non-conditioned medium which supports,ES cell growth in the absence of LIF.

The source of the undifferentiated ES cell is as described above.

The non-conditioned medium used in combination with the human feeder cell layer is important for supporting the undifferentiated cell growth, in particular for deriving new cell lines in an undifferentiated state. Preferably, the human feeder cells are obtained from human adult fallopian tubal or human embryonic muscle and skin feeder cells, human fetal muscle or skin or adult oviductal epithelial fibroblasts or skin cells. More preferably, the human feeder cells are fetal muscle cells or adult skin cells.

The non-conditioned medium is kept simple by the absence of LIF, mostly hLIF which is generally supplemented to the culture when mouse feeder cells are used.

Media which supports ES cell growth may be selected from the group including HES, KO, HES-HS or KO-HS.

For prolonged propagation and derivation of ensuing undifferentiated human embryonic stem (HES) cells to produce a cell line, HFM, HFS or HAFT feeder layers may be used together with any one of the four (4) media above that are used to grow ICMs. HES-HS and KO-HS are preferred because they do not contain animal based ingredients.

In yet another aspect of the present invention, there is provided a cellular composition comprising proliferating undifferentiated ES cells which are free of feeder cells and wherein the cell composition comprises the propagated or derived ES cells prepared by the methods described herein in the absence of feeder cells.

Preferably the cell compositions of the present invention are provided as a cell culture wherein the cells are cultured on a cell support matrix comprising a component or a combination of components selected from the group including but not limited to Collagen I, Collagen IV or Matrigel. Most preferably, the component or components include Collagen I or Matrigel. In the absence of feeder cells, the cultured undifferentiated ES cells are sustained in conditioned media, as described herein.

In yet another aspect of the present invention, there is provided a cellular composition comprising proliferating undifferentiated ES cells on a feeder cell layer wherein the cell composition comprises the propagated or derived ES cells prepared by the methods described herein.

The feeder cell layer comprises human cells and are preferably selected from the group including but not limited to human adult fallopian tubal or human embryonic muscle and skin feeder cells, human fetal, muscle or skin or adult oviductal epithelial fibroblasts or skin.

Most preferably, the feeder layers in order of preference are adult skin cells, embryonic muscle, embryonic skin and adult fallopian tubal cells. More preferably they are adult skin cells or fetal muscle cells, most preferably human fetal muscle cells. They are preferably grown on plastic ware without collagen.

In another aspect of the present invention there is provided a human feeder cell layer which supports ES cells in culture in a substantially undifferentiated state.

Applicants have found few sources of feeder cells which can support the ES cells preferably human ES cells, in a substantially undifferentiated state when in culture. The feeder cells may be used in a similar way to known fibroblast feeder cell layers which are presently used to support and sustain ES cells in a substantially undifferentiated state. Preferably, the feeder cell layer comprises human cells or muscle or skin feeder cells from an embryo or fetus. Adult feeder cells may also be used. Most preferably, they are human fetal muscle feeder cells or they may be human adult fallopian tubal cells or adult oviduct epithelial fibroblasts or adult skin cells.

The embryonic skin and muscle may be derived from normal non-pathological 14 week old abortuses. The tissues may be specifically dissected from these areas. The adult human fallopian tubal cells may be derived from non-pathological pre-menopausal fallopian tubes donated by women who are undergoing sterilization. The cells may be collected from the inner epithelial lining of the fallopian tubes. Preferably, the cells are all screened for HIV, hepatitis B and other pathogens. Muscle, skin and tubal cells are all processed by the same method as described in Bongso et al (1989) Hum Reprod, 4;486-494. Whilst these cells are directly derived from their natural sources, it is also considered that the cells may derive from sustained cultures of these feeder cells.

Preferably, the ES cells are human ES cells and the feeder layer comprises human cells.

The feeder layers are preferably cultured in the presence of a HF medium which may contain 10% FCS, but most preferably, the FCS is replaced by 10% HS (HF-HS). Alternatively, the feeder cells are established as primary cultures in the presence of a HFE medium. To maintain the cells by serial passaging a human maintenance (HM) medium may be used. However, before passaging, the cells must be detached from the culture surface. EDTA with mechanical agitation or the use of a rubber policeman may facilitate dislodgment and reduce the need for non-human based proteases such as trypsin.

Feeder cells may also be selected from the group including Detroit 551, MRC-5 or WI-38.

In yet another aspect of the present invention, there is provided an undifferentiated ES cell prepared by the methods described herein, most preferably, the ES cell is a mammalian ES cell. More preferably it is a primate cell selected from monkey or human. Even more preferably, the ES cell is a human ES cell.

In yet another aspect of the present invention there is provided an ES cell line derived by the methods described herein.

The ES cells and ES cell lines described herein may be defined by any markers used to characterise ES cells. These include, but are not limited to, the expression of Oct-4, TRA-1-60, proteoglycans, SSEA and SCID mice teratoma production.

In another aspect of the present invention there is provided a cell culture system for providing ES cultures in a substantially undifferentiated state, said system comprising:

a cell support matrix; and

a cell culture medium for providing soluble factors derived from human feeder cells or equivalents thereof.

The system is designed to be used to culture ES cells in a substantially undifferentiated state. The ES cells may be obtained from any source as described above.

The cell support matrix may be supported on any surface of a suitable culture system which supports cell growth. Ideally the cell support matrix is produced on a tissue culture plate which may be of any suitable composition, preferably plastic or glass. Other suitable surfaces include slides, glass beads, gelatin-coated plastic ware, albumin coated plasticware, polylysine coated plasticware, biodegradable polymers used as scaffolds by bioengineers and marine adhesives.

The cell support matrix may be any of the cellular or non-cellular support matrices described above. Most preferably they are human cellular or non-cellular support matrices.

The culture medium may be any medium which supports ES cell growth. Preferably it is a conditioned medium as described above. However, where the cell support matrix is a feeder cell layer as herein described, a non-conditioned medium may be used for receiving soluble factors produced from feeder cells of the cell support matrix. Preferably, the non-conditioned medium is as described above which is also absent of LIF or hLIF.

The cell culture system may be provided as a kit for use with the methods described herein.

In a preferred aspect of the present invention, there is provided a cell culture system including:

a cell support matrix comprising collagen I or matrigel; and

a conditioned medium including soluble factors derived from a human feeder cell layer.

In an even more preferred aspect of the present invention, there is provided a cell culture system including:

a cell support matrix comprising collagen I; and

a conditioned medium including soluble factors derived from a human feeder cell layer selected from the group including embryonic muscle, skin or adult fallopian tubal feeder layer, fetal muscle and skin fibroblasts, adult oviduct epithelial fibroblasts or adult skin cells.

More preferably, the conditioned medium is derived from a feeder layer comprising embryonic muscle, or skin cells. More preferably, the feeder layer is a human feeder layer.

In yet another preferred aspect of the present invention, there is provided a cell culture system for deriving and propagating an ES culture in a substantially undifferentiated state, said system comprising:

a cellular cell support matrix comprising human feeder cells; and

a non-conditioned cell culture medium absent LIF for supporting culture of the ES cells.

The human feeder cell layer is as described above. It is preferred that the cell culture system is used to receive undifferentiated ES cells from sources as described above to derive new cell lines of ES cells and to maintain or sustain ES cultures which have already been cultured.

The medium used in the cell culture system is preferably a medium which supports ES cell growth. As described above, such media include HES, KO, HES-HS or KO-HS. HES-HS and KO-HES are most preferred because they do not contain animal based ingredients.

The methods described herein for culturing ES cells in a substantially undifferentiated state will be seen to be applicable to all technologies for which ES cells are useful. Of particular importance is the creation of new cell lines for propagation. It is possible that inner cell masses (ICMs) from human blastocysts can be isolated by immunosurgery and propagated in an undifferentiated state on the human feeder layers and non-cellular matrices with or without conditioned media as described herein. Also, it provides for cell lines having single or multiple genetic modifications.

Genetic modifications are desirable for many reasons such as providing modified genes for gene therapy or replacement of tissues for grafting or implantation.

Methods used to perform the genetic modifications to the cells can be any of those known in the field of molecular biology for making such genetic transformations.

The term “genetic modification” as used herein includes alternations to the sequence encoding a gene product, as well as alterations to flanking regions, in particular the 5′ upstream region of the coding sequence (including the promoter). Similarly, the term “gene” encompasses the coding sequence and regulatory sequences that may be present flanking the coding sequence, as well as other sequences flanking the coding sequence. In addition, as is known in the art, genetic modifications can be achieved by introducing a nucleic acid that does not necessarily comprise the entire gene sequence into the cell, e.g., by introducing a nucleic acid that can be inserted into the genome by recombination.

Much attention recently has been devoted to the potential applications of stem cells in biology and medicine. The properties of pluripotentiality and immortality are unique to ES cells and enable investigators to approach many issues in human biology and medicine for the first time. ES cells potentially can address the shortage of donor tissue for use in transplantation procedures, particularly where no alternative culture system can support growth of the required committed stem cell. ES cells have many other far reaching applications in human medicine, in areas such as embryological research, functional genomics, identification of novel growth factors, and drug discovery, and toxicology.

Thus, the present invention provides novel materials and methods for growing ES cells in a substantially undifferentiated state. Using the methods and materials provided by the present invention ES cells, such as those isolated from humans and monkeys, can be grown more efficiently. The ability to grow efficiently such cells without differentiation has important applications for therapeutic uses of ES cells for treating human diseases using tissue transplantation and/or gene therapy techniques where such cells are used directly or following one or more genetic modifications as described herein. In addition, ES cells grown using the methods and materials described herein can be used to screen for new bioactive substances or for other factors that promote or retard the differentiation of such cells in culture.

The present invention will now be more fully described with reference to the following examples. It should be understood, however, that the description following is illustrative only and should not be taken in any way as a restriction on the generality of the invention described above.

### EXAMPLES

### Example 1:

Growth of HES Cells on Collagen I and Matrigel TM.

a) Preparation of Conditioned Media

90% confluent monolayers of passage 4 MEF, passage 3 human embryonic muscle, human embryonic skin (8 week old abortuses) and passage 6 adult premenopausal human fallopian tubal fibroblast cells grown in a T-75 (Falcon, USA) tissue culture flask were treated with mitomycin-C (Sigma, M-0503) for 2.5 hours at 37° C., 5% CO₂ in air. The monolayer was dispersed with 0.05% Trypsin-EDTA to produce a single-cell suspension of feeder cells. The trypsin-EDTA was removed by centrifugation and separation of supernatant and the pellet of feeder cells were then plated on gelatin-coated plastic 1 ml single well tissue culture dishes (Falcon, USA). 180,000 mitomycin-C treated feeder cells were plated on each 1 ml dish. Next, each dish was washed twice with fresh HES medium and the washed inactivated feeder cells incubated overnight in the presence of HES medium for 16 hours at 37° C., 5% CO₂ in air. The 16 hour conditioned HES medium was then collected, filtered using a 0.22 μm filter (Sterivex, Millipore) and used fresh or stored at 4° C. to be later warmed to 37° C. and used.

b) Preparation of Non-conditioned Media

Non-conditioned medium comprised of 80% Dulbecco's Modified Eagles Medium (Life Technologies, cat no. 11960-044), 20% Hyclone defined Fetal Calf Serum (Hyclone cat no. SH 30070.03), 1× L-Glutamine (Life Technologies, cat no. 25030-081), 1× Penicillin/Streptomycin (Life Technologies, cat no. 15070-063), 1× Non-essential Amino Acids (Life Technologies, cat no. 11140-050), 1× Insulin-Transferrin-Selenium G supplement (Life Technologies, cat no. 41400-0450), 1 mM β-mercaptoethanol (Life Technologies, cat no. 21985-023).

c) Preparation of Coatings on Tissue Culture Plates

(i) BIOCOAT® Collagen I Cellware 35 mm petri dishes from Becton Dickinson (Cat no. 40456) were used in all experiments. The manufacturer's source of collagen I was from the rat tail tendon (Becton Dickinson product catalog).

(ii) COLLAGEN I liquid was from Becton-Dickinson (Cat no. 40231). The source was bovine dermis and plating of plasticware was at 10 ug/cm² of tissue culture surface.

(iii) MATRIGEL™ precoated plasticware was from Becton Dickinson (Cat no. 40460). The source of matrigel was engelbreth-holm-swarm mouse tumour.

d) Growth of HES Cells on Collagen I and Matrigel™ with Conditioned Media

(i) Experimental group: Mature undifferentiated HES-3 and HES4 cell colonies are grown according to patent application PCT/AU99/00990 and are grown on MEF cells and were cut with a sterile needle into fragments of about 300 cells each, lifted from the feeder layers with Dispase (Sigma cat no. P3417) and transferred to 35 mm collagen I and matrigel coated petri dishes (Becton Dickinson). HES cells were grown on this matrix in these dishes in the presence of 2 ml of 16 hour 0.22 μm Sterivex® (Millipore) filtered feeder (MEF/embryonic muscle, skin/adult fallopian tubal) conditioned HES medium. Conditioned medium was changed daily and cells were grown at 37° C. in 5% CO₂ in air.

(ii) Control group: Similar sized fragments of undifferentiated HES cells from the same cell lines (HES-3 and HES-4) were lifted from the feeder layers with Dispase (Sigma) and re-grown on (1) plain plastic dishes with feeder conditioned medium, (2) collagen I coated dishes with HES medium.

Both experimental and control dishes were housed in the same incubators and monitored daily for 7 days for colony formation and growth (differentiated or undifferentiated). Several replicates were attempted. Two different HES cell lines (HES-3 and HES-4) from different ethnic backgrounds were evaluated.

e) Growth of HES Cells on Human Embryonic Muscle, Skin and Adult Human Fallopian Tubal Cells with Non-conditioned HES Medium

(i) Experimental group: Mature undifferentiated HES-3 and HES-4 cell colonies grown on MEF cells were cut with a sterile needle into fragments of about 300 cells each, lifted from the MEF layers with Dispase (Sigma cat no. P3417) and transferred to 1 ml single well tissue culture dishes (Falcon, USA) containing plated (˜180,000 mitomycin-C treated) human embryonic muscle, skin fibroblasts and human adult fallopian tubal fibroblast feeder cells in the presence of normal non-conditioned HES medium.

(ii) Control group: Similar sized fragments of undifferentiated HES cells from the same cell line (HES-3 and HES-4) were lifted from the feeder layers with Dispase and re-grown on mitomycin-C treated MEF feeder layers.

Both experimental and control dishes were housed in the same incubators and monitored daily for 7 days for colony formation and growth (differentiated or undifferentiated). Several replicates were attempted. Two different HES cell lines (HES-3 and HES-4) from different ethnic backgrounds were evaluated.

f) Growth of HES Cells on the Human Fetal Skin Fibroblast Cells Detroit 551

The Detroit 551(CCL-110) cell line is from the skin of a female Caucasian fetus. It has a normal karyotype and has a finite lifespain of 25 serial subcultures from the tissue of origin. It is shipped from the ATCC at the 10^{th} passage. The culture was expanded in vitro with HF medium to the 14^{th} passages and cryopreserved. Confluent monolayers of passage 14 or 15 Detroit 551 fibroblast cells grown in HF medium were treated with mitomycin-C (Sigma, St. Louis, Mo.) for 2.5 hours. The monolayer was dispersed to produce a single-cell suspension of feeder cells and plated on gelatin-coated plastic 1 ml single well tissue culture dishes (Falcon, Becton Dickinson, USA). 180,000 mitomycin-C treated D551 cells were plated on each 1 ml dish.

Detroit 551 cells are capable of supporting prolonged undifferentiated HES cell growth in vitro. HES 3 and the new cell line colonies grown on Detroit 551 feeders appear morphologically similar to undifferentiated HES colonies grown on HFM, HFS and HAFT fibroblast feeders. HES colonies on D551 have straight edges like colonies that form on other human fibroblast feeders and individual HES cells under high power magnification display prominent nucleoli with a high nuclear to cytoplasm ratio.

g) Confirmation of Undifferentiation

Several methods of characterization were used.

(1) Morphological characteristics

Daily observation of HES cell morphology under bright field and phase contrast inverted optics was carried out. Particular attention was paid to the speed of growth in an undifferentiated state, to the morphology of the inner, outer and edges of each colony for homogeneity of cell size, thickness and tightly packed nature of colonies, sharpness and reflective nature of colony edges, spreading nature of colonies and nuclear-cytoplasmic cell ratios.

(2) RT-PCR

Fragments of colonies from both experimental and control dishes were separated with dispase, washed and then frozen for characterization by RT-PCR.

(3) SCID mice

Fragments (15-20) were also taken from each experimental and control dish and injected into SCID mice to produce teratomas in 6 to 8 weeks. Teratomas were then separated and processed for conventional histology to confirm presence of human tissues from all three primary germ lineages.

h) Summary

Preliminary data suggests that HES cells are able to attach, spread and grow well maintaining an undifferentiated state in the presence of MEF, human adult fallopian tubal and human embryonic muscle and skin feeder fibroblast conditioned HES medium on collagen I (precoated as well as liquid coated) and matrigel precoated plastic petri dishes. Furthermore, mitomycin-C treated human adult fallopian tubal feeders, human embryonic muscle feeders and human embryonic skin feeders are also able to support undifferentiated HES cell growth in the presence of non-conditioned HES medium.

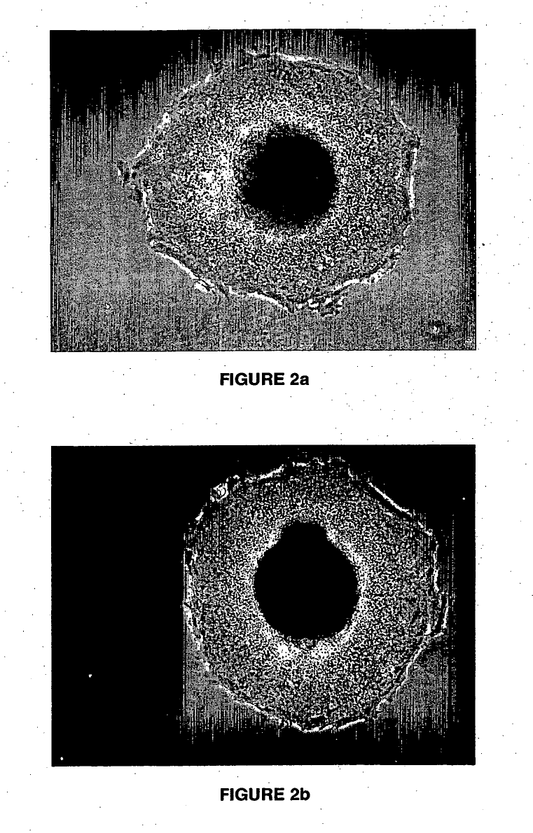

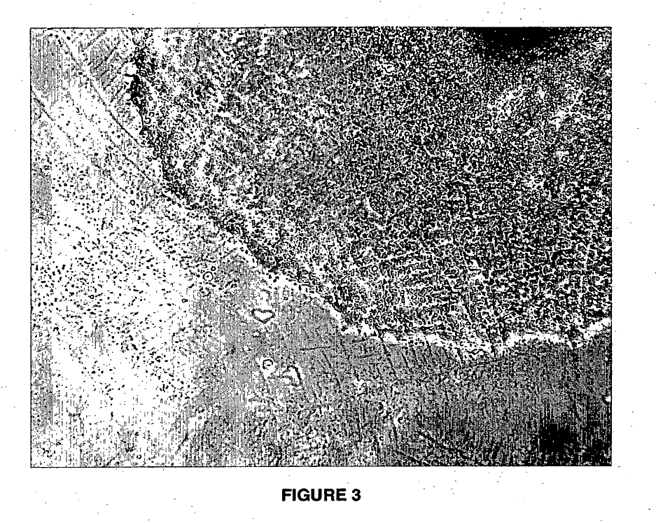

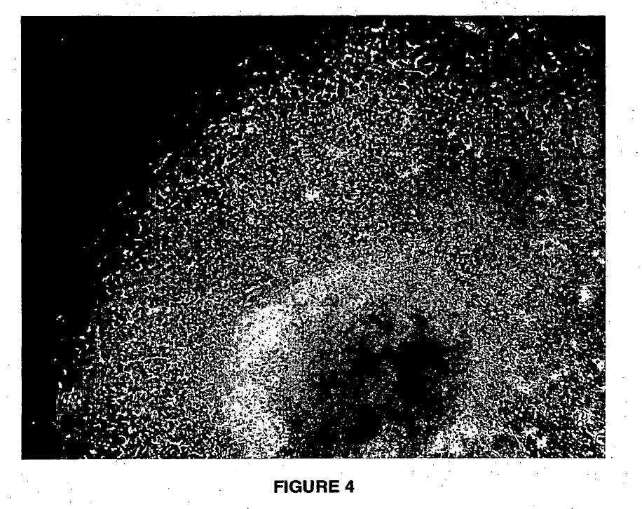

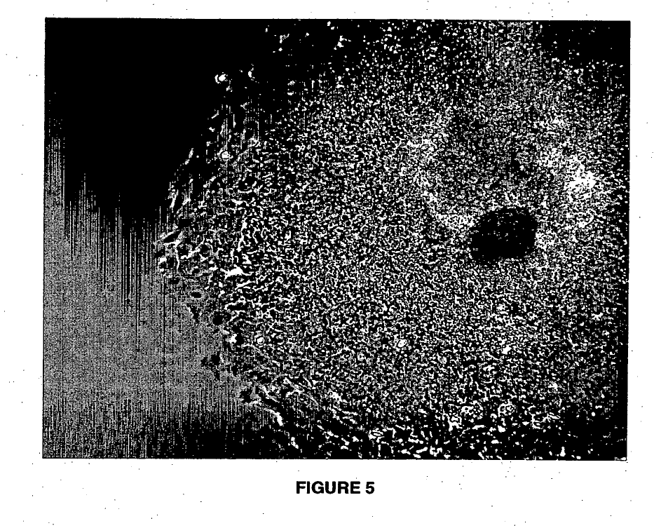

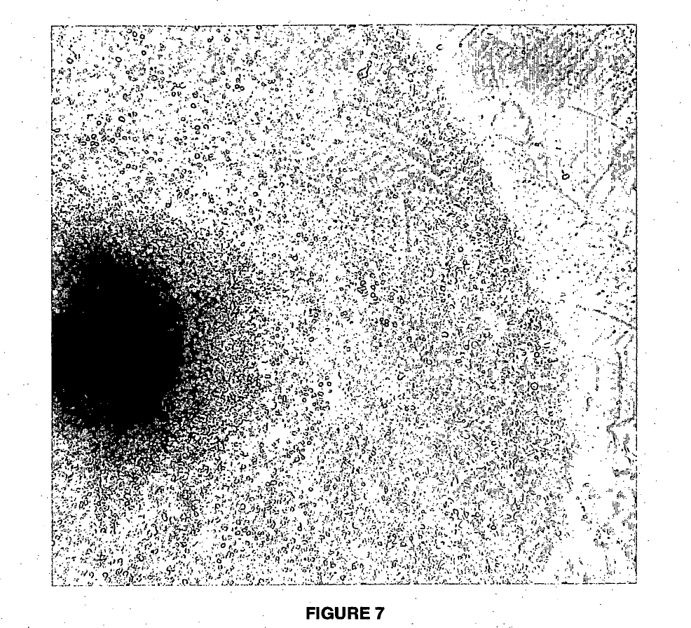

HES cell colonies grown on the collagen I dishes with feeder layer conditioned media maintain a tightly packed morphology similar to that of pluripotent HES cells grown on MEF feeders even after 7 days in culture (see FIGS. 2a, 2b, 3, 4, and 5). HES cells grown on the collagen I coated plastic also display high nuclear-cytoplasmic mass ratio typical of pluripotent HES cells grown on inactivated mouse feeders. Although colony growth on matrigel coated dishes was as good as on collagen I, the ES cells on matrigel spread out more rapidly, are thinner and as such difficult to passage (see FIG. 7).

Physical attachment to the MEF cells does not appear to be critical for the survival of HES cells. However, feeder layer conditioned HES medium seems essential for the growth and maintenance of the undifferentiated state when HES cells are grown on collagen I or matrigel coated dishes Differentiation occurred when HES cells were grown in the presence of non-conditioned HES medium on collagen I and matrigel matrices and also when HES cells were grown on plastic with feeder layer conditioned medium. HES cells grown on collagen I can be cut into smaller fragments, lifted with the metalloprotease, dispase and transferred to fresh dishes for continued propagation in vitro.

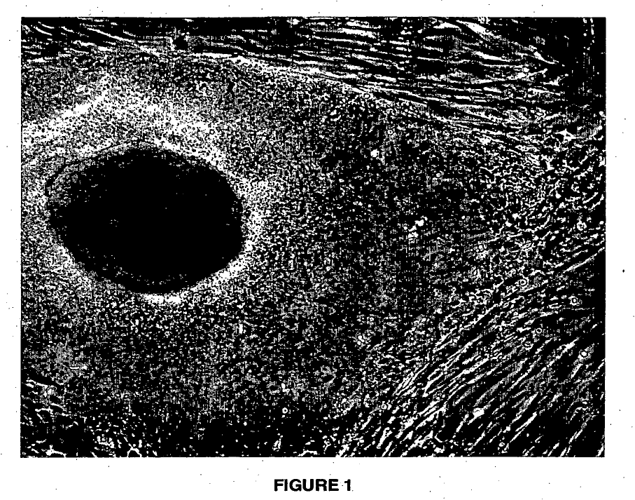

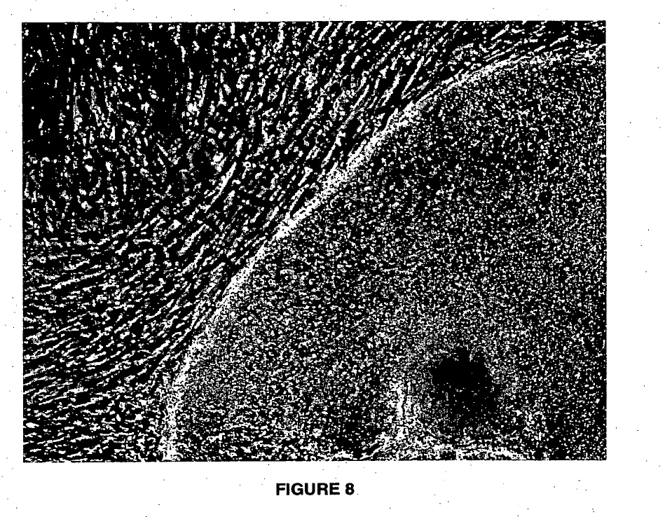

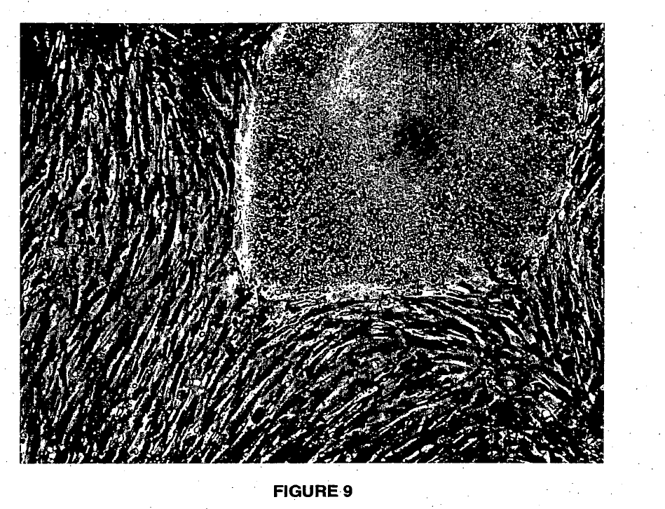

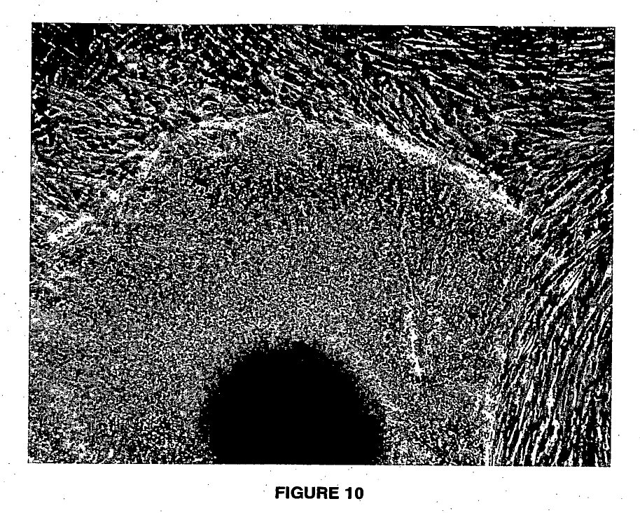

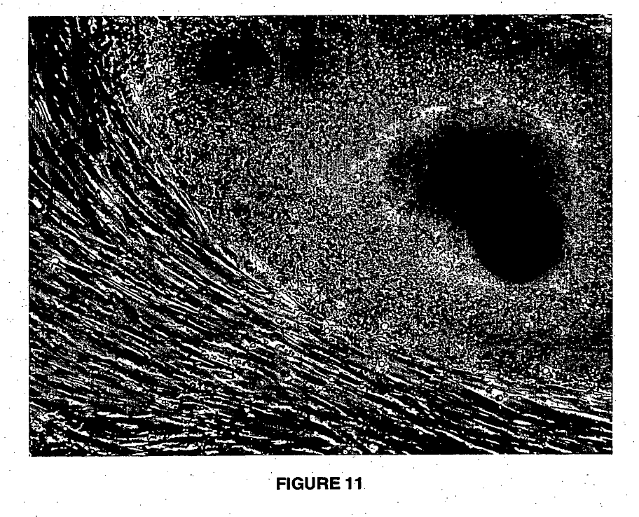

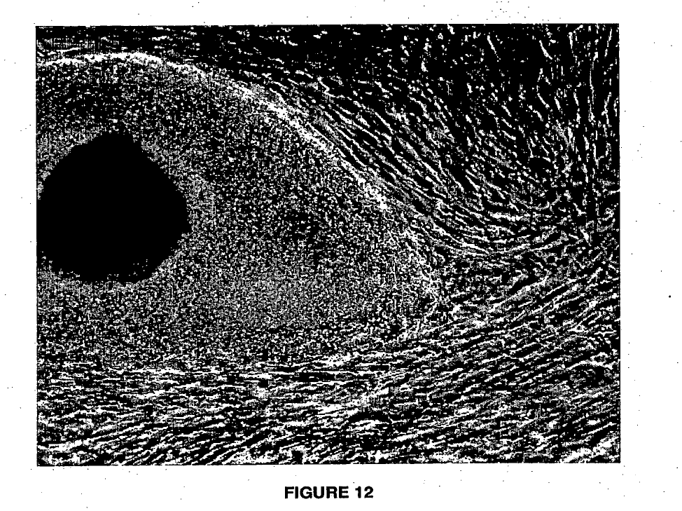

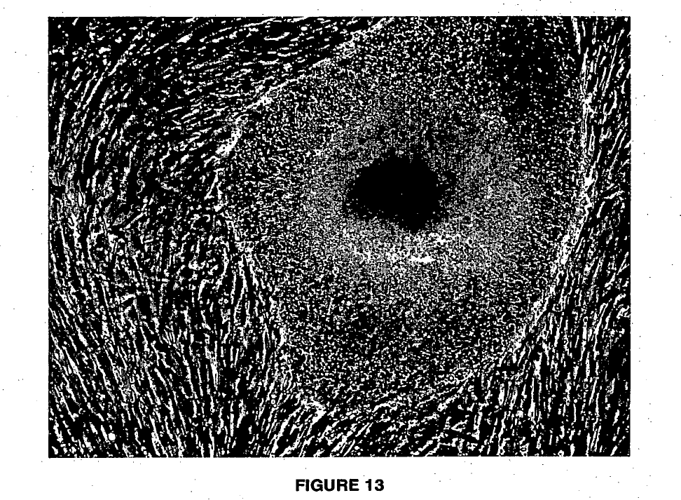

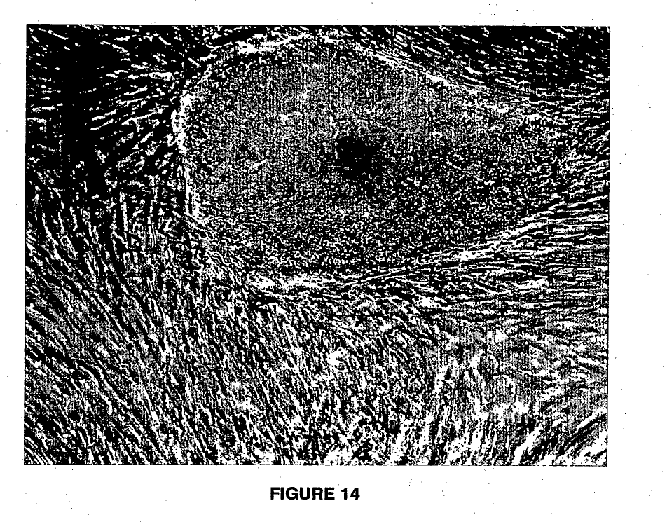

HES cells also form colonies and remain undifferentiated when grown directly on human embryonic muscle, skin and human adult fallopian tubal feeder fibroblasts (see FIGS. 1, 8, 9, 10, 11, 12, 13 or 14). HES cells growing on collagen I coated dishes with MEF, human embryonic muscle, skin and human adult fallopian tubal feeders can also grow well in an undifferentiated state.

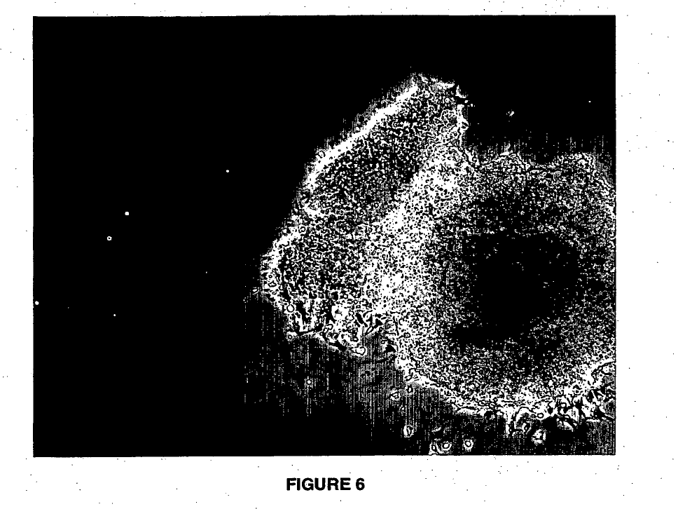

ES cells cannot grow in an undifferentiated state on Laminin coated culture dishes (FIG. 6), in the presence of conditioned media.

SCID mice and RT-PCR studies are underway to ascertain if HES cells grown on collagen I and the human feeder layers are indeed pluripotent.

### Example 2:

Growth of Human ICMs and Derivation of Ensuing Human Embryonic Stem Cells on Human Feeder Layers

a) Human Feeder Layer Preparation

Human fetal muscle samples were obtained directly from the thigh muscle of fresh normal 14 week human abortuses. The fetal muscle samples were mechanically cut into very fine pieces with pointed curved sterile scissors in transport medium (ASP 100, Vitrolife,Goteborg, Sweden) in a sterile plastic Petri dish. The explants and cell suspension were centrifuged at 300 g for 10 mins, supernatant decanted and the pellet containing explants and cells seeded into 25 cm² sterile plastic tissue culture flasks containing 2 ml of Chang's medium (Irvine Sc, Calf, USA) or human feeder establishment medium (HFE) and incubated at 37° in a 5% CO₂ in air atmosphere. After 1 week primary cultures (fibroblasts) were established.

The muscle fibroblasts were detached from the plastic by trypsinization with trypsin-EDTA (GIBCO, Grand Island, USA). Alternatively, they may be treated with EDTA with mechanical agitation or by using a rubber policeman. This avoids the use of trypsin for cell dissociation. Once detached the cells are centrifuged, supernatant decanted and cell pellets seeded into new 25 cm² tissue culture flasks containing DMEM medium (GIBCO) supplemented with 10% FBS (GIBCO) or 10% to 20% human serum 1× L-glutamine (GIBCO) and penicillin-streptomycin (GIBCO). This DMEM supplemented medium is now called HES medium (Human embryonic stem cell medium). Continuous subculturing was carried out once confluency was obtained at each subculture up to the 6^{th} passage. Muscle fibroblasts from 4^{th}, 5^{th} and 6^{th} subcultures in DMEM supplemented medium were frozen in liquid nitrogen (−196° C.).

For growth and support of human inner cell masses (ICMs) and ensuing human ES cells, passage 4, 5 or 6 muscle fibroblasts were first thawed and seeded in tissue culture flasks and grown until they became confluent.

The muscle fibroblast cultures were treated with Mitomycin C and once they were 95% confluent the mitomycin C was washed away and cells plated on 1-well organ culture dishes at a density of 180,000 cells per dish.

b) Immunosurgery to Separate ICMs from Frozen-thawed Human Embryos

A 2-day old frozen embryo was thawed and grown in vitro with G 2.2 sequential medium (Vitrolife, Goteborg, Sweden) until the day-6 blastocyst stage. Only good quality blastocysts with large inner cell masses (ICMs) are used.

The blastocyst was incubated in Pronase (10 IU) (Protease, Sigma, Mo., USA) (prepared in G 2.2 medium) for 2 mins at 37° C. in a 5% CO₂ in air atmosphere to remove the zona pellucida.

After complete zona removal, the blastocyst was washed thoroughly in Dulbecco's Phosphate Buffered Saline (DPBS) to remove the G 2.2 medium and incubated with Anti-Human Whole Serum (1:1 dilution with DBPS) for 30 min at 37° C. in 5% CO₂ in air.

Following incubation with Anti-Human Whole Serum, the blastocyst was washed thoroughly with DPBS again and incubated with Guinea Pig Complement (1:1 dilution with DP BS) at 37° C. in 5% CO₂ in air for 30 min.

The blastocyst was then transferred to HES medium (DMEM supplemented) and passed through a fine drawn out polished glass Pasteur pipette several times to release and separate the Inner Cell Mass (ICM) clump.

The ICM clump of cells was washed thoroughly in HES medium before plating on to the Mitomycin C inactivated human muscle feeder layers.

c) ICM Plating

Medium in the 1-well dish was changed 3 times with fresh HES medium before transferring the ICM to the human feeder layer. The ICM broke into 2 or 3 small clumps. Each clump was placed separately on the feeder layer. The feeder layer was prepared 2 days before plating ICMs. The dishes containing the ICM on feeder layers were carefully incubated without disturbance to allow the ICM to attach and grow on the feeder layer. Daily monitoring of the ICM growth under inverted phase contrast optics was carried out. The medium was changed to fresh HES medium 2 or 3 times in the first week.

d) Sub-culture of ICM Primary Cultures

The first subculture of the ICM was performed 7 days after initial plating when an area of small, round ES-like cells with a prominent nucleolus was observed.

The medium in the dish was changed twice with DPBS and the sharp edge of a 30 G sterile needle was used to cut around the perimeter of the clump of ES-like cells and the clump itself was cut into 2 equal pieces.

DPBS was removed and 1 ml of filtered Dispase solution (Sigma, Mo., USA) at a concentration of 0.17 g/10 ml was added to the dish.

After an incubation period of 30 seconds, each half of the ES clump was carefully removed using a Gilson P20 micropipette with a sterile pipette tip and transferred to a new 1-well dish with Mitomycin C inactivated human fetal muscle feeder cells containing HES medium.

Medium in the new dish was changed daily after each clump attached to the feeder layer.

Growth of the ES cell clumps was also monitored daily.

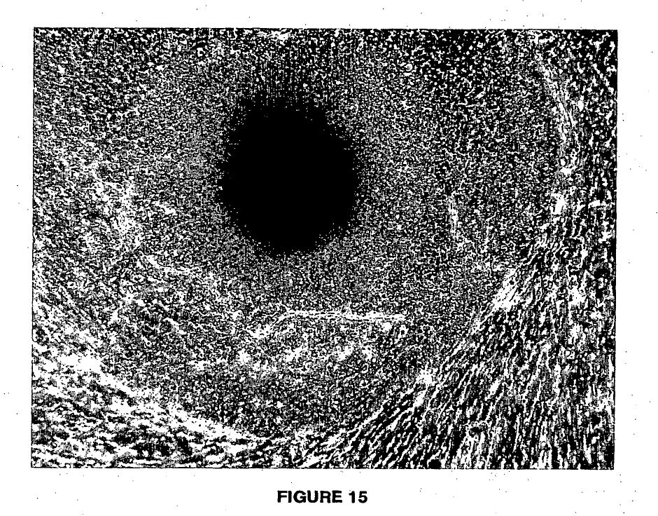

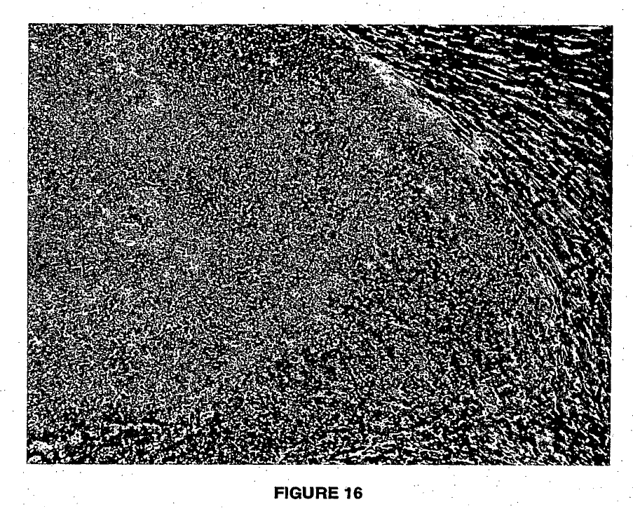

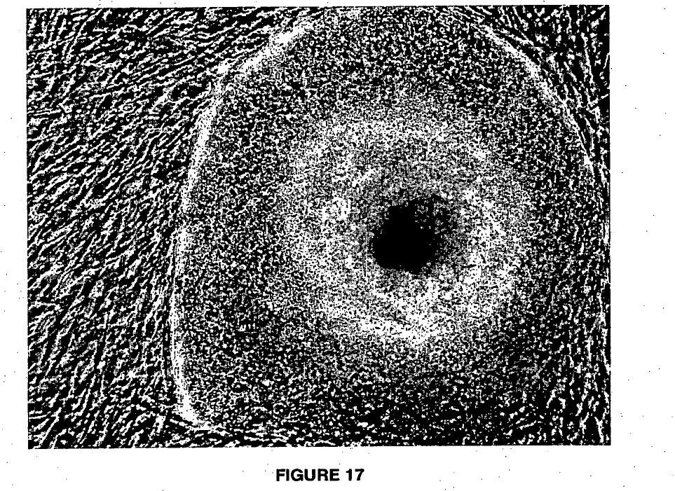

After 8 to 10 days, the clumps of ES cells matured into typical ES cell-like colonies. The undifferentiated regions of the colonies were sub-cultured again on new fetal muscle feeder layers. Continuous subculturing was carried out in this way. FIG. 15 shows a colony of undifferentiated HES cells at the 17^{th} passage. FIG. 16 shows the edge of the colony.

e) Characterization with Oct-4 RT-PCR

Cells were characterized by the methods outlined in Example 1. For determination of Oct4-RT-PCR, the following primers were used:

Forward primer:

CGRGAAGCTGGAGAAGGAGAAGCTG

Reverse primer:

CAAGGGCCGCAGCTTACACATGTTC

Expected product: 247 bp

f) Other Characterization Methods

The undifferentiated HES cells from the ICM grown on human feeders (10^{th} passage) expressed Oct-4, were SSEA and Tra-1-60 positive by immunostaining, displayed normal Giemsa banded karyotypes and showed all three primary germ layers (pluripotent) in the teratomas in SCID mice.

The HES cell line derived is supported by human fetal muscle feeders and has survived to the 19^{th} passage to date. This cell line retains all the typical morphological characteristics of other HES cell lines supported by mouse embryonic fibroblast feeders such as colony growth, sharp and defined colony boundaries in undifferentiated colonies and small round ES-like cells with a prominent nucleolus. Like all other HES cell lines derived to date, this cell line also tests positive for Oct-4 expression. Morphologically, this HES cell line forms thinner colonies than other HES cell lines supported by mouse feeders.

Finally, it is to be understood that various other modifications and/or alterations may be made without departing from the spirit of the present invention as outlined herein.

1. A human feeder cell layer which supports the derivation of ES cells in a substantially undifferentiated state said feeder cell layer comprising cells selected from the group including human adult, fetal or embryonic cells or a combination thereof.

2. A human feeder cell layer which supports the culture of ES cells in a substantially undifferentiated state said feeder cell layer comprising cells selected from the group including human adult, fetal or embryonic cells or a combination thereof.

3. A human feeder cell layer according to claim 1 or 2 wherein the human adult cell is selected from the group including human fibroblast cells, human adult skin and human adult muscle fibroblasts and adult epithelial cells or a combination thereof.

4. A human feeder cell layer according to claim 3 wherein the human adult cell is a human fibroblast cell.

5. A human feeder cell layer according to claim 3 wherein the human fibroblast cell is a human adult fallopian tubal (HAFT) fibroblast cell.

6. A human feeder cell layer according to claim 3 wherein the human adult cell is a human skin cell.

7. A human feeder cell layer according to claim 3 wherein the human adult cell is a human muscle cell.

8. A human feeder cell layer according to claim 3 wherein the human adult cell is a human adult epithelial cell.

9. A human feeder cell layer according to claim 8 wherein the human adult epithelial cell is a human oviductal epithelial cell.

10. A human feeder cell layer according to claim 1 or 2 wherein the human fetal cell is a human fetal muscle (HFM) or human fetal skin (HFS) cell or combination thereof.

11. A human feeder cell layer according to claim 10 wherein the human fetal cell is a HFM cell.

12. A human feeder cell layer according to claim 10 wherein the human fetal cell is a HFS cell.

13. A human feeder cell layer according to claim 1 or 2 wherein the human embryonic cell is a human embryonic muscle (HEM) or human embryonic skin cell or combination thereof.

14. A human feeder cell layer according to claim 13 wherein the human embryonic cell is a HEM cell.

15. A human feeder cell layer according to claim 13 wherein the human embryonic cell is a human embryonic skin cell.

16. A human feeder cell layer according to claim 1 or 2, which is first established in a primary culture in the presence of HFE medium.

17. A human feeder cell layer according to claim 16 wherein the feeder layer is propagated in the presence of a HM medium.

18. A human feeder cell layer according to claim 1 or 2 comprising fibroblast cell line Detroit 551 (ATCC NO CCL-110).

19. A human feeder cell layer according to claim 1 or 2 comprising cell line MRC-5 having accession Number ATCC No. X-55 or ATCC No. CCL-171.

20. A human feeding layer according to claim 1 or 2 comprising cell line WI-38 having Accession Number ATCC NO CCL-75 or ATCC NO CCL-75.1.

21. A method of deriving an embryonic stem (ES) cell line in a substantially undifferentiated state from an ES cell population said method comprising:
obtaining an ES cell population comprising undifferentiated ES cells; and
culturing the undifferentiated ES cells on a cell support matrix in the presence of soluble factors derived from human feeder cells or equivalents thereof.

22. A method according to claim 21 wherein deriving an ES cell line is selected from the group including creating an ES cell line from a source of ES cells and wherein the ES cells are previously uncultured cells; extending propagation or culturing time of an ES cell line wherein the ES cell line is an established cell line; and propagating an established ES cell line.

23. A method according to claim 22 wherein the deriving of the ES cell line includes propagating an ES cell line.

24. A method according to any claim 21 wherein the ES cell population is derived from a source selected from the group including an embryo, blastocyst, inner cell mass (ICM) cells, and a culture of ES cells which have not differentiated.

25. A method according to claim 24 wherein the source is from a blastocyst.

26. A method according to claim 21 wherein the soluble factors are derived from human feeder cells selected from the group including human adult, fetal or embryonic cells or a combination thereof.

27. A method according to claim 26 wherein the human adult cell is selected from the group including human fibroblast cells, human adult skin and human adult muscle fibroblasts and adult epithelial cells or a combination thereof.

28. A method according to claim 27 wherein the human adult cell is a human fibroblast cell.

29. A method according to claim 27 wherein the human fibroblast cell is a human adult fallopian tubal (HAFT) fibroblast cell.

30. A method according to claim 27 wherein the human adult cell is a human skin cell.

31. A method according to claim 27 wherein the human adult cell is a human muscle cell.

32. A method according to claim 27 wherein the human adult cell is a human adult epithelial cell.

33. A method according to claim 27 wherein the human adult cell is a human oviductal epithelial fibroblast.

34. A method according to claim 26 wherein the human fetal cell is a human fetal muscle (HFM) or human fetal skin (HFS) cell or combination thereof.

35. A method according to claim 34 wherein the human fetal cell is a HFM cell.

36. A method according to claim 34 wherein the human fetal cell is a HFS cell.

37. A method according to claim 26 wherein the human embryonic cell is a human embryonic muscle (HEM) or human embryonic skin cell or combination thereof.

38. A method according to claim 37 wherein the human embryonic cell is a HEM cell.

39. A method according to claim 37 wherein the human embryonic cell is a human embryonic skin cell.

40. A method according to claim 21 wherein the human feeder cells are cultured in the presence of a medium selected from a group including HES, KO, HF, HES-HS, KO-HS, and HF-HS as hereinbefore described.

41. A method according to claim 40 wherein the medium is HES-HS or KO-HS.

42. A method according to claim 41 wherein the medium is KO-HS.

43. A method according to claim 21 wherein the cell support matrix is a non-cellular cell support matrix selected from the group including Collagen I, Collagen IV, human extracellular matrix or Matrigel or a combination thereof.

44. A method according to claim 43 wherein the cell support matrix comprises Collagen I or Type I Collagen.

45. A method of deriving an embryonic stem (ES) cell line in a substantially undifferentiated state from an ES cell population said method comprising:
obtaining an ES cell population comprising undifferentiated ES cells;
and culturing the undifferentiated ES cells on a cell support matrix in the presence of soluble factors derived from human feeder cells or equivalents thereof, wherein the cell support matrix comprises a human feeder cell layer according to claim 1 or 2.

46. A method according to claim 45 wherein the ES cells are cultured in the presence of a medium selected from the group including HES, KO, HES-HS, KO-HS and HF-HS as hereinbefore described.

47. A method according to claim 46 wherein the medium is KO-HS.

48. A method according to claim 45 wherein the feeder cells are first established in primary cultures in the presence of HFE medium, as hereinbefore described.

49. A method according to claim 45 wherein the feeder cells are propagated in the presence of a HM medium prior to culture with ES cells, as hereinbefore described.

50. A method according to claim 45 wherein the human feeder cell is the fibroblast cell line Detroit 551 (ATCC NO CCL-110).

51. A method according to claim 45 wherein the human feeder cell is the cell line MRC-5 having Accession Number ATCC No. X-55 or ATCC No CCL 171.

52. A method according to claim 45 wherein the human feeder cell is the cell line W1-38 having Accession Number ATCC-CCL-75 or ATCC-CCL-75.1.

53. A method according to claim 45 wherein the ES cell line is cultured in the absence of LIF.

54. A cellular composition comprising proliferating undifferentiated ES cells and wherein the cell composition comprises the propagated or derived ES cells prepared by the methods according to claim 21.

55. An undifferentiated ES cell line prepared by a method according to claim 21.

56. A cell culture system for deriving and culturing ES cells in a substantially undifferentiated state, said culture system including:
a cell support matrix; and
a cell culture medium for providing soluble factors derived from a human feeder cell selected from the group including a human adult, fetal or embryonic cell.

57. A cell culture system according to claim 56 wherein the human adult cell is selected from the group including human adult fallopian tubal (HAFT) fibroblast cells, human adult skin and human adult muscle fibroblasts and adult epithelial cells or a combination thereof.

58. A cell culture system according to claim 57 wherein the human adult cell is a human fibroblast cell.

59. A cell culture system according to claim 57 wherein the human adult cell is a human adult fallopian tubal (HAFT) fibroblast cell.

60. A cell culture system according to claim 57 wherein the human adult cell is a human skin cell.

61. A cell culture system according to claim 57 wherein the human adult cell is a human muscle cell.

62. A cell culture system according to claim 57 wherein the human adult cell is a human adult epithelial cell.

63. A cell culture system according to claim 57 wherein the human epithelial adult cell is a human oviductal epithelial cell.

64. A cell culture system according to claim 56 wherein the human fetal cell is a human fetal muscle (HFM) or human fetal skin (HFS) cell or combination thereof.

65. A cell culture system according to claim 64 wherein the human fetal cell is a HFM cell.

66. A cell structure system according to claim 64 wherein the human fetal cell is a HFS cell.

67. A cell culture system according to 56 wherein the human embryonic cell is a human embryonic muscle (HEM) or human embryonic skin cell or combination thereof.

68. A cell culture system according to claim 67 wherein the human embryonic cell is a HEM cell.

69. A cell culture system according to claim 67 wherein the human embryonic cell is a human embryonic skin cell.

70. A cell culture system according to claim 56 wherein the cell support matrix comprises Collagen I or matrigel or a combination thereof.

71. A cell culture system according to claim 70 wherein the cell support matrix comprises Collagen I.

72. A cell culture system according to claim 56 wherein the cell culture medium is a conditioned medium including soluble factors derived from a human feeder cell layer.

73. A cell culture system for deriving and culturing ES cells in a substantially undifferentiated state, said culture system including:
a cell matrix; and
a cell culture medium for providing soluble factors derived from a human feeder cell selected from the group including a human adult, fetal or embryonic cell, wherein the cell support matrix comprises a human feeder cell layer according to claim 1 or 2.

74. A cell culture system according to claim 56 wherein the culture medium is selected from the group including HES, KO, HES-HS, and KO-HS.

75. A cell culture system according to claim 74 wherein the medium is KO-HS.

76. A conditioned medium for deriving and culturing ES cell line in a substantially undifferentiated state said medium prepared by a method including:
obtaining a feeder cell layer according to claim 1 or 2;
culturing the feeder cells in the presence of a medium selected from the group including HES, KO, HES-HS, KO-HS, HFE, HM, HF, or HF-HS; and
separating the medium from the cells to obtain conditioned medium.

77. A conditioned medium according to claim 76 wherein the human feeder cell layer comprises adult skin cells.

78. A conditioned medium according to claim 77 wherein the human feeder cell layer comprises HFM cells.

79. A conditioned medium according to claim 76 wherein the medium comprises KO-HS.
